(19) United States
(12) Patent Application Publication
(10) Pub. No.: US 2012/0058942 A1
(43) Pub. Date: Mar. 8, 2012

(54) METHODS FOR CONTROLLING BLOOD-GLUCOSE LEVELS IN INSULIN-REQUIRING SUBJECTS

(76) Inventor: John DUPRE, London (CA)

(21) Appl. No.: 13/223,724

(22) Filed: Sep. 1, 2011

Related U.S. Application Data
(60) Provisional application No. 61/380,000, filed on Sep. 3, 2010.

(51) Int. Cl. A61K 38/28; A61P 3/10; A61K 38/22

(52) U.S. Cl. 514/6.5; 514/7.2

(57) ABSTRACT

The present invention relates to methods of controlling blood glucose levels in subjects receiving an insulin therapy. The methods include administering to the subject receiving an insulin therapy a dipeptidyl peptidase inhibitor and a GLP-1 related peptide. The present invention relates also to the use of such a combination for treating insulin-requiring diabetes mellitus and conditions related thereto.

This application claims the benefit of the filing date of U.S. Provisional Application No. 61/380,000, filed Sep. 3, 2010 by the present inventor, the content of which is specifically incorporated herein by reference.

### FIELD OF THE INVENTION

The present invention relates to methods for controlling blood-glucose levels in insulin-requiring subjects.

### BACKGROUND OF THE INVENTION

I. Complications of Diabetes Mellitus:

With the establishment of therapy with insulin in insulin-deficient diabetes in humans the complications of long-term diabetes mellitus were recognized in what is now termed Type 1 diabetes (T1D). It was later recognized that similar complications occur in long-term maturity onset diabetes, now known as Type 2 diabetes (T2D). The metabolic control achievable with exogenous insulin in these early decades following discovery of the hormone did not restore blood glucose levels to the normal range and it was speculated that long-term hyperglycaemia had a mechanistic role in the development of the microvascular and macrovascular complications associated with long-term diabetes.

Development of Intensive Insulin Therapy for Type 1 Diabetes. In the 1990s multiple daily injections or continuous subcutaneous infusions of insulin were shown to enable hitherto unattainable degrees of near-normalization of blood glucose levels under everyday conditions through long periods of time (1). These programs, now known as intensive insulin therapies (IIT), made it possible to address the question whether such further improvement in glycaemic control would reduce the rates of complications affecting the eyes, kidneys, nerves and major blood vessel systems in people with insulin-treated diabetes.

In the 1980s it was demonstrated that large-scale multi-centre international clinical trials of IIT are feasible and can be undertaken with acceptable safety (2). The first ever trial on a scale large enough to test the hypothesis was undertaken with the support of the US NIH in 27 centres (24 in the USA and 3 in Canada) comparing the long-term effects of IIT with those of then-conventional standard clinical use of insulin (1 or 2 injections a day). The trial, known as the Diabetes Control and Complications Trial (DCCT), was initiated in 1982 and was successful in maintaining a very substantial difference in the mean blood glucose levels in more than 1400 volunteers who were randomly assigned to conventional therapy or to HT. By 1993 the DCCT Study Group was able to report that IIT resulted in a reduction in the risks of the complications in long-term insulin-treated diabetes by 50% or more (3). The protocols employed in this study became the standard of care for insulin-requiring subjects with phenotypic T1D ie. those subjects recognized to have diabetes mellitus associated with very severe or absolute deficiency of endogenous insulin secretion.

Continuing experience with IIT in T1D has shown that strict normo-glycaemia cannot be achieved because of the risk of intermittent hypoglycaemia, and because of the risk of progressive obesity, so that compromise levels of glycaemic control have been accepted during IIT as currently performed, virtually world-wide. Thus it is accepted that ‘normo-glycaemia’ is not attainable by means of available therapy. The evidence, however, continues to suggest that the degree of hyperglycaemia that has to be tolerated as a result of this therapeutic compromise is largely responsible for the continuing though reduced morbidity and excess mortality of diabetes (4).

Intensive Insulin Therapy for Type 2 Diabetes. With respect to insulin-requiring phenotypic T2D, it is recognized that conclusions drawn from studies in volunteers with phenotypic T1D may not be applicable to subjects with phenotypic T2D. This clinical form of diabetes is well recognized as generally developing at a later age. It is associated with obesity in many subjects. However, it was inferred that benefits of IIT would be demonstrable in people with phenotypic T2D (3). It is clear that a very high proportion of people with long-term phenotypic T2D (more than 75%) eventually develop hyperglycaemia of a degree that calls for insulin treatment, and this has become the standard progression in treatment of T2D with diet, oral hypoglycaemic agents, and later as glycaemic control fails, with addition of administration of insulin according to the same programs used in T1D. Thus IIT is indicated in clinical practice for the management of T2D when clinical-guideline-approved targets for glycaemic control are not attainable without administration of insulin. The adverse effects of IIT remain prominent obstacles to its optimal use in T1D or T2D. It is clear that the major adverse effects of IIT, ie. intermittent hypoglycaemia, and progressive weight gain, constitute an obstacle to the acceptance of IIT. These risks and the wide-spread fear of them are responsible for reluctance on the part of physicians as well as patients to initiate IIT in T2D, where it is optional, rather than obligatory as it is in T1D.

II. Recent Development of Therapeutic Agents with Potential for Use in Insulin-Requiring Diabetes:

For more than 100 years there has been interest in gastro-intestinal functions that appear to be involved in regulation of intermediary metabolism and of blood glucose levels (5,6). An endocrine component of this system, served by hormones secreted by the gastro-intestinal tract in response to ingestion of nutrients, was hypothesized. Interest in it was revived in the 1960s when the present inventor and colleagues coined the term ‘entero-insular axis’ to cover these functions (7). There followed a search for the insulin-stimulating agents of this entero-insular axis, with interest in the possibility that failure of the axis might contribute to the pathophysiology of diabetes mellitus, and that identification of agents of the axis might reveal potential therapeutic approaches.

In 1973 the present inventor and his colleagues reported their identification of such an insulin-stimulating hormone, which was then named gastric-inhibitory polypeptide and given the acronym GIP (8). Further research showed that deficiency of GIP was not a major factor in the pathophysiology of diabetes in humans, and that administration of GIP failed to stimulate insulin secretion in humans with diabetes mellitus even among those who had substantial residual endogenous insulin production. It also became clear that there was more than one hormonal agent of the entero-insular axis. Glucagon-like peptide-1 (GLP-1) was then identified as a post-translational product of the glucagon gene in the intestinal L-cell, which proved to be capable of stimulating insulin secretion in people with residual insulin-producing cells and with manifest diabetes mellitus (9). Like GIP, GLP-1 is secreted in response to arrival of nutrient in the small intestine and thus is recognized as an incretin.

Subsequent work in two approaches to the development of therapeutic agents has borne fruit in providing for improvement of glycaemic control in humans (10). The first of these recent approaches is based on the use of the incretins, which were first tested in T2D. These agents represent the natural hormones or analogs, which can serve to improve insulin secretion in people with T2D. The agents have been classed as incretins after the physiological term given to the natural hormones of the entero-insular axis. These insulin-stimulating hormones have a very short half-life in the circulation. This brevity of action is due to the endogenous dipeptidyl peptidases, a complex system with multiple enzymes that destroy peptides of this class very quickly. Therefore a second approach to developing therapies based on the incretin system has been to follow the effects of synthetic dipeptidyl peptidase inhibitors (10). Work on inhibitors of dipeptidyl peptidase (DPPI), also known as gliptins, has yielded oral formulations that are effective in prolonging the life of endogenous incretins and apparently thereby in improving glycaemic control in people with T2D. It is notable that the pharmaceutical companies producing incretins have indicated that their use should be confined to non-insulin-receiving subjects and indeed that their use in insulin-treated subjects is contra-indicated in the product monographs.

III. Potential Therapeutic Value of Incretins and/or DPPIs in Insulin-Treated Diabetes in Humans

Prompted by findings from studies undertaken by the inventor in human volunteers with recent-onset T1D mellitus, who show evidence of residual, but sub-normal, insulin secretory capacity, studies were undertaken in insulin-treated T1D during continued administration of insulin by IIT. The inventor found that the incretin GLP-1 (in its physiological forms as GI ,P-1 7-37 or as GLP-1 7-36 amide) can powerfully restrict the rise in blood glucose that accompanies absorption of a standard meal (11). This effect was not associated with change of insulin secretion or change of insulin clearance and appeared to be independent of delivery of endogenous insulin. It was accompanied by evidence of delay in absorption of the meal and by evidence of suppression of secretion of glucagon, the hormone that stimulates glucose output from the liver. Pursuit of further understanding of these phenomena lead the inventor to obtain US Pat. No. 6,899,883 covering use of GLP-1 as a congener with insulin in treatment of T1D.

A number of diabetes therapies have been developed using long acting GLP-1 analogs and agonists. However, the use of GLP-1 analogues or agonists involves exposure to continuing pharmacological blood levels of a non-human agent with unknown long-term risks and widespread effects on multiple enzymes involved in degradation of dipeptidases. Among these risks is the occurrence of acute pancreatitis, now documented in subjects receiving long-acting GLP-1 agonist and/or DPPIs (12). The available data regarding this risk do not permit conclusions as to the contributions from the components of the combined therapy, but it is understood that the addition of a ‘blackbox’ warning of this risk to the product monograph is under consideration DPPIs raise the blood levels of both incretins, and of other proteins or peptides that may have non-desired effects. Moreover blood levels of the incretin GIP are also elevated and this can result in adverse effects through stimulation of glucagon secretion (13). The risks of the available agents can be minimized by reduction of dosage and/or intermittent administration of these agents, which can be expected to reduce the risk of tachyphylaxis, and that of provocation of an immune response with or without neutralization of the action of the peptide.

Recent studies in dogs by Bergman and colleagues suggest that there are direct effects of

GLP-1 or GLP-1 receptor agonists on glucose clearance from the circulation, probably through actions on the liver and in the muscles, depending for expression on adequacy of delivery of insulin, but not involving changes in delivery or clearance of insulin (14).

In summary, the apparently counter-intuitive suggestion that insulin-stimulating hormones may be useful in the control of glycaemia in people with severe insulin deficiency in the form of T1 D has been validated empirically. It is clear that safer and more effective means of improving glycaemic control (and other aspects of metabolic control) in both Type 1 and T2D are necessary for realization of the potential benefits of reduction of the long-term risks of the complications of diabetes through improved control.

### SUMMARY OF THE INVENTION

The present invention relates to a therapy for controlling blood glucose levels in subjects undergoing an insulin therapy.

As such, in one embodiment, the present invention relates to a method of controlling blood glucose level in a subject receiving an insulin therapy comprising administering to the subject: (i) a dipeptidyl peptidase inhibitor (DPPI); and (ii) a glucagon-like petide-1 (GLP-1) related peptide.

The present invention relates also to methods of optimizing therapeutic efficacy for controlling blood glucose level in a subject receiving an insulin therapy. As such, in another embodiment, the present invention provides for a method of optimizing therapeutic efficacy for controlling blood glucose level in a subject receiving an insulin therapy comprising: a. establishing a dose of insulin according to insulin intensive therapy guidelines; b. administering a DPPI to the subject; c. reducing the dose of insulin administered to the subject; and d. administering a GLP-related peptide to the subject.

The present invention relates also to uses of insulin, DPPI and GLP-1 related peptide. As such, in another embodiment, the present invention provides for a use of (i) a dipeptidyl peptidase inhibitor (DPPI); (ii) a GLP-1 related peptide and (iii) insulin for controlling blood glucose level in a subject receiving an insulin therapy comprising administering to the subject.

### DETAILED DESCRIPTION OF THE INVENTION

1. Definitions

The term “blood glucose level” or “blood GLP-I level” shall mean blood glucose concentration or blood GLP-1 concentration, respectively. In certain embodiments, blood GLP-1 level is a level in blood of biologically active GLP-I, wherein GLP-I having agonist activity at GLP- IR is biologically active. In certain embodiments, a blood glucose level or blood GLP-I level is a plasma glucose level or a plasma GLP-I level.

The term “elevated blood glucose level” shall mean an elevated blood glucose level such as that found in a subject demonstrating clinically inappropriate basal and postprandial hyperglycaemia or such as that found in a subject in oral glucose tolerance test (oGTT).

The term “insulin-requiring” means a subject that requires insulin treatment in accordance with current guidelines, such as those of the American Diabetes Association, providing recommendations for the management of diabetes (15).

The term “controlling blood glucose level” shall mean bringing and/or maintaining blood glucose levels within the limits defined in current therapeutic guidelines.

The term “subject,” as used herein, shall refer to a mammal, including but not limited to a mouse, a rat, a rabbit, a pig, a dog, a cat, a non-human primate and a human, more preferably to a mouse or rat, most preferably to a human.

In this specification, the term “GLP-1 related peptides” includes, without limitation, GLP-1(7-37), GLP-1(7-36) amide, analogues of GLP-1(7-37) or of GLP-1(7-36) amide, peptides which differ from GLP-1(7-37) or from GLP-1(7-36) amide in that at least one of the amino acid residues of GLP-1(7-37) or of GLP-1(7-36) amide has been exchanged by another amino acid residue, preferably one which can be coded for by the genetic code. The definition also comprises the case when amino acid residues are added at or deleted from the N-terminal and/or the C-terminal end of GLP-1(7-37) or GLP-1(7-36) amide. Preferably, the total number of such additions, deletions and exchanges does not exceed 5, more preferred it does not exceed 3. The definition also includes agonists of GLP-1(7-37) or GLP-1(7-36) amide, such as exendin-4.

2. Overview

As mentioned above, subjects with diabetes treated with diet and oral agent or agents may gradually fail to respond to insulin therapy.

Furthermore, insulin requiring subjects treated with DPPIs or with long lasting forms of GLP-1 may suffer the risk of developing acute pancreatitis and other unknown long-term risks, discussed above (12). IIT's adverse effects include progressive weight gain and intermittent hypoglycaemia episodes, which constitute an obstacle to the acceptance of IIT.

Thus, the present invention, in one embodiment, provides for a method of controlling blood glucose level in a subject receiving an insulin therapy comprising administering to the subject: (i) a dipeptidyl peptidase inhibitor (DPPI); and (ii) a glucagon-like petide-1 (GLP-1) related peptide. The insulin, DPPI, GLP-1 related peptide and insulin may be administered to the subject separately.

The dose of the DPPI, the dose of the GLP-1 related peptide and the dose of insulin in the combination therapy of the present invention may be smaller than the dosages of the DPPI, GLP-1 related peptide, or insulin when used individually for controlling blood-glucose level in subjects with diabetes mellitus. Reduction in the dosage and/or intermittent administration of these agents may reduce, minimize or avoid the risk of adverse effects due to these agents. Notably, the dosage with GLP-1 agonists required in combination with IIT is much lower than that defined with their use in the absence of IIT, so that the common symptomatic adverse effects of nausea and vomiting produced by these agents can be avoided.

Given that pharmaceutical companies producing incretins have indicated that the use of incretins should be confined to non-insulin-receiving people, the methods of the present invention are indeed, surprising. The concomitant treatment with a combination of a DPPI, a GLP-1 related peptide, and insulin, may result in a synergistic response by the insulin-requiring subject that gives rise to controlling blood glucose level using dosages of insulin, DPPI and GLP-1 that will minimize or avoid the side effects of these agents. Combined treatment with DPPI, GLP-1 related peptides, and insulin is thus novel, therapeutically useful, and surprising. Unforeseen, therapeutic advantages may be gained by treating the insulin-requiring subjects with all three types of drugs.

The GLP-1 related peptides may be administered by methods currently available according to the invention for administration of peptides, including, without limitation, nasal, oral, intravenous. Nasal application is particularly advantageous from a patient compliance point of view.

The GLP-1 related peptide may be administered in a dosage which may achieve about 50% or more of attainable reduction of blood glucose level after a meal. Preferably, the GLP-1 related peptide may administered in a dosage range that is equivalent to or less than the therapeutic dose range of said GLP-1 related peptide when used alone for controlling blood glucose level in an insulin-requiring subject with diabetes mellitus. In aspects of the invention, the dose of GLP-1 related peptide may be between about 7 nanograms to about 40 nanograms daily. In aspects of the invention the GLP-1 related peptide may be administered in the GLP-I related peptide manufacturer's recommended dosage. Less than the manufacturer's recommended dosage may also be used.

The DPPI agent used according to the invention can be administered, for example, orally. I lowever, other forms of administration may be possible. Any DPPI exhibiting a glucose lowering effect may be used in the combination of the present invention. One example of a DPP-4 inhibitor is JANUVIA™ (sitagliptin, MERK™). The DPPI may be administered in a dosage to achieve about 40% to about 60% inhibition of the level of dipeptidyl peptidase activity in the subject. Preferably, the DPPI may be administered in a dosage range that is equivalent to or less than the therapeutic range of DPPI used in the absence of insulin for controlling blood glucose levels in a subject with diabetes mellitus. In aspects of the invention, the dose of DPPI peptide may be about 100 mg daily or less. In aspects of the invention the DPPI may be administered in the DPPI manufacturer's recommended dosage (see for example http://www.merck.com/product/usa/pi_circulars/j/januvia/januvia_pi.pdf). Less than the manufacturer's recommended dosage may also be used.

Insulin can be administered according to any known method and in accordance with therapeutic guidelines.

The combination of the present invention results in a greater and more selective enhancement of the actions of the incretin, by comparison with the effects of more powerful and broader-acting inhibition of DPPI. It should be noted that the gradual loss of weight experienced by subjects with T1D or T2D during treatment with GLP-1 agonists is independent of other effects of the agonist: it is limited to a few kilograms and constitutes an advantage of this component of the combination therapy of the present invention (12) in overcoming the weight gain that commonly accompanies IIT in treatment of diabetes.

The rapid degradation of GLP-1 may be inhibited by using low doses of clinically approved DPPIs which may maintain blood levels of GLP-1 at levels sufficient to recruit the effects of delayed intestinal absorption and of inhibition of glucagon secretion, together with the direct metabolic effects of the incretin on glucose disposal, to enable the combination therapy proposed for insulin-requiring diabetes mellitus. Thus, the net effect of addition of the GLP-1 related peptide during IIT may be a substantial improvement in insulin sensitivity, as originally defined by studies of blood glucose levels in insulin-treated diabetes mellitus (16).

In one embodiment, the present invention relates also to methods of treating an insulin-requiring subject with diabetes mellitus. In one aspect of the present invention, the method of treating an insulin-requiring subject may comprise administering the subject with a DPPI, a GLP-I related peptide and insulin. The combination of DPPI, GLP-1 related peptide and insulin may provide an effect in controlling a blood glucose level in the subject. The dosages of the DPPI, the GLP-1 related peptide, and the insulin in the therapy of the present invention may be smaller than that of the DPPI, GLP-1 related peptide or insulin when used individually to reduce blood-glucose levels in subjects with diabetes mellitus.

The present invention discloses also methods for personalized medicine. As such, in one embodiment, the present invention provides for a method of optimizing therapeutic efficacy for controlling blood glucose level in a subject receiving an insulin therapy comprising, comprising: a.establishing a dose of insulin according to insulin therapy guidelines; b. administering a DPPI to the subject; c. reducing the dose of insulin administered to the subject; and d. administering a GLP-related peptide to the subject.

Insulin therapy guidelines may be those provided by The American College of Physicians (ACP). A recommended target for blood glucose level may be 7.8 to 11.1 mmol/L (140 - 200 mg/dL) if insulin is used, employing these numbers to define the acceptable range between 7.8 mmol/L fasting and 11.1 mmol/L after meals, respectively.

The features disclosed in the present description, examples and claims may, both separately and in any combination thereof, be material for realizing this invention in diverse forms thereof. The invention is further illustrated by the following examples which are not to be construed as limiting, but merely as an illustration of some preferred features of the invention.

### EXAMPLES

The inventor and colleagues have shown that the dose-response relationships of therapies are very similar in T1D and T2D with continuation of IIT in both categories of disease (16). This finding enables establishment of the methods and uses of the present invention according to available dose-response information with guidelines provided for instituting the proposed combination therapy, without the need for quantification of endogenous insulin secretion or determination of the presence of indicators of auto-immune diabetes as the basis of insulin requirement under clinical conditions.

The proposed combination therapy of the present invention may be initiated in any subject requiring insulin therapy when treatment according to current therapeutic guidelines fails to establish a targeted glycaemic control. Thus, the situation in which the proposed therapies may be initiated is one in which the subject is receiving and maintaining insulin therapy (by insulin pump or multiple daily injection programs), but where blood glucose control is evidently outside the targeted range (haemoglobin ale determinations exceeding normal by unacceptable degrees in accordance with current guidelines). Under these conditions the initiation of the proposed therapies may be implemented as a first step by establishment of dosage of DPPI inhibitor targeting approximately 50% inhibition of the dipeptidase (this dosage is indicated in the product description of the inhibitor and in instructions for its use). With continued monitoring of blood glucose control according to best clinical practice, in a suitable formulation to be administered, for example by subcutaneous injection with insulin before each significant meal will follow. This will usually involve dosage of GLP-1 related peptide in the range that has been shown to achieve more than 50% of the attainable reduction of glycaemic excursions after meals. Further adjustment of GLP-1 related peptide may be taken to the maximum dose that avoids symptomatic adverse effects and avoids hypoglycaemia. Furthermore, through this process which is familiar in the management of insulin-treated diabetes, the insulin dosage may be reduced where possible thus reducing the risk of hypoglycaemia. In this process the established daily insulin dosage with insulin intensive therapy may be initially reduced (ie. with introduction of one or both of the congeners) by 10% or more with continuing downward adjustment in light of blood glucose levels and haemoglobin Alc determinations, in order to minimize insulin dosage and risk of hypoglycaemia. Such dose adjustments in relation to continuing blood glucose monitoring are familiar to people managing HT, both those with diabetes, and their care-givers.

The risks described above for the use of long acting GLP-1s and/or DPPIs can be minimized with the therapy herein proposed through intermittent administration of the incretin with avoidance of continuous action and with minimization of dosage of DPPI afforded by provision of exogenous incretin.

### REFERENCES

1. Dupre J. Open loop continuous insulin infusion in management of insulin requiring diabetes mellitus. IN: The Diabetes Annual/1. Edited by KCMM Alberti. Elsevier Science BV 1985:129-151.
2. The Kroc Collaborative Study Group. Collaborative studies of the effects of continuous subcutaneous insulin infusion in insulin dependent diabetes mellitus: Conclusions. Diabetes 1985;34[Suppl 3]:87 89.
3. The DCCT Research Group. The effect of intensive treatment of diabetes on the development and progression of long-term complications in insulin-dependent diabetes mellitus. N Engl J Med 1993;329(14):977-986.
4. The DCCT Research Group. Effect of glycemic exposure on the risk of microvascular complications in the Diabetes Control and Complications Trial—revisited. Diabetes 2008;57:995-1001.
5. Conard V. Acta gastroenterol Belg 1955;18:655.
6. Dupre J. An intestinal hormone affecting glucose disposal in man. Lancet 1964;2:672-673.
7. Unger R H, Ketterer H, Dupre J, Eisentraut A M. The effects of secretin, pancreozymin and gastrin on insulin and glucagon secretion in anesthetized dogs. J Clin Invest 1967;46(4):630-645.
8. Dupre J, Ross S A, Watson D, Brown J C. Stimulation of insulin secretion by gastric inhibitory polypeptide in man. J Clin Endocrinol Metab 1973;37(5):826-828.
9. Kreymann B, Williams G, Ghatei M A, Bloom S R. Glucagon-like peptide-1 7-36: a physiological incretin in man. Lancet 1987;2:1300-1304.
10. Ahren B, Schmitz 0. GLP-1 receptor agonists and DPP-4 inhibitors in the treatment of type 2 diabetes. Horm Metab Res 2004;36:867-876.
11. Dupre J, Behme M T, McDonald T J. Exendin-4 normalized postcibal glycemic excursions in Type 1 diabetes. J Clin Endocrinol Metab 2004;89(7):3469-3473.
12. Nauk M A, Meier J J. Individualized incretin-based treatment for Type 2 diabetes. Lancet 2010;376:393-394.
13. Dupre J, Caussignac Y, McDonald T J, Van Vliet S. Stimulation of glucagon secretion by gastric inhibitory polypeptide in patients with hepatic cirrhosis and hyperglucagonemia. J Clin Endocrinol Metab 1991;72(1):125-9.
14. Zheng D, lonut V, Mooradian V, Stefanovski D, Bergman R N. Exenatide sensitizes insulin-mediated whole-body glucose disposal and promotes uptake of exogenous glucose by the liver. Diabetes 2009;58(2):352-359.
15. American Diabetes Association. Clinical Practice Recommendations. Diabetes Care 2010;33[Suppl 1]:S1-S100.
16. Dupre J, McDonald T J. Therapeutic potential of glucagon-like peptide-1 agonists in insulin-treated diabetes mellitus. [abstract P-1734 ]. Can J Diabetes. 2009;33:319.
17. Ahren B, et al. Twelve- and 52-week efficacy of the dipeptidyl peptidase IV inhibitor LAF237 in metformin-treated patients with type 2 diabetes. Diabetes Care 2004;27:2874-2880.

1. A method of controlling blood glucose level in a subject receiving an insulin therapy comprising administering to the subject: (i) a dipeptidyl peptidase inhibitor (DPPI);
and (ii) a glucagon-like petide-1 (GLP-1) related peptide.

2. The method of claim 1 wherein the insulin, DPPI, GLP-1 related peptide and insulin are administered to the subject separately.

3. The method of claim 1 wherein the subject has type 1 or type 2 diabetes mellitus.

4. The method of claim 1 wherein the DPPI is produced by a manufacturer, and the DPPI is administered in the manufacturer's recommended dosage.

5. The method of claim 1 wherein the DPPI is administered in a dose range that is equivalent to or less than the therapeutic dose range of DPPI when used individually for controlling blood glucose level in an insulin-requiring subject with diabetes mellitus.

6. The method of claim 1 wherein the DPPI is administered in a dosage that targets between about 40% and about 60% of the inhibition of the level of dipeptidyl peptidase in the subject.

7. The method of claim 1 wherein the DPPI is administered in a dosage of about 100 mg daily or less.

8. The method of claim 1 wherein the GLP-1 related peptide is administered in a dosage which achieves about 50% or more of attainable reduction of blood glucose level after a meal.

9. The method of claim 1 wherein the GLP-1 related peptide is administered in a dosage range that is equivalent to or less than the therapeutic dose range of said GLP-1 related peptide when used alone for controlling blood glucose level in an insulin-requiring subject with diabetes mellitus.

10. The method of claim 1 wherein the dose of GLP-1 related peptide is between about 7 nanograms to about 40 nanograms daily.

11. The method of claim 1 wherein the GLP-1 related peptide is produced by a manufacturer, and the GLP-1 related peptide is administered in the manufacturer's recommended dosage.

12. The method of claim 1 wherein the GLP-1 related peptide is selected from GLP-1 7-37 or GLP-1 7-36 amide.

13. The method of claim 1 wherein the insulin dose in the insulin therapy is equivalent to the therapeutic dose range of said insulin when used individually for controlling blood glucose level in an insulin-requiring subject with diabetes mellitus.

14. The method of claim 1 wherein the insulin dose in the insulin therapy is less than the therapeutic dose range of said insulin compound when used alone for controlling blood glucose level in an insulin-requiring subject with diabetes mellitus.

15. A method of optimizing therapeutic efficacy for controlling blood glucose level in a subject receiving an insulin therapy comprising, comprising:
a. establishing a dose of insulin according to insulin therapy guidelines;
b. administering a DPPI to the subject;
c. reducing the dose of insulin administered to the subject; and
d. administering a GLP-related peptide to the subject.

16. The method of claim 15 wherein the subject has type 1 or type 2 diabetes mellitus.

17. The method of claim 15 wherein in said method further comprises monitoring blood glucose level of the subject, and wherein a decrease or an increase in the level of blood glucose indicates a need to adjust the dose of insulin administered to said subject.

18. The method of claim 15 wherein the DPPI is produced by a manufacturer, and the DPPI is administered in the manufacturer's recommended dosage.

19. The method of claim 15 wherein the DPPI is administered in a dose range that is equivalent to or less than the therapeutic dose range of DPPI when used individually for controlling blood glucose level in an insulin-requiring subject with diabetes mellitus.

20. The method of claim 15 wherein the DPPI is administered in a dosage that targets between about 40% and about 60% of the inhibition of the level of dipeptidyl peptidase in the subject.

21. The method of claim 15 wherein the DPPI is administered in a dosage of about 100 mg daily or less.

22. The method of claim 15 wherein the GLP-1 related peptide is administered in a dosage which achieves about 50% or more of attainable reduction of blood glucose level after a meal.

23. The method of claim 15 wherein the GLP-1 related peptide is administered in a dosage range that is equivalent to or less than the therapeutic dose range of said GLP-1 related peptide when used individually for controlling blood glucose level in an insulin-requiring subject with diabetes mellitus.

24. The method of claim 15 wherein the GLP-1 related peptide is administered in a dosage of between about 7 nanograms to about 40 nanograms daily.

25. The method of claim 15 wherein the GLP-1 related peptide is produced by a manufacturer, and the GLP-1 related peptide is administered in the manufacturer's recommended dosage.

26. The method of claim 15 wherein the GLP-1 related peptide is selected from GLP-1 7-37 or GLP-1 7-36 amide.
